(19) United States
(12) Patent Application Publication
(10) Pub. No.: US 2024/0018053 A1
(43) Pub. Date: Jan. 18, 2024

(54) CERAMIC SUBSTRATE AND PREPARATION METHOD FOR THE SAME, CERAMIC HEATING BODY, AND ELECTRONIC VAPORIZATION DEVICE

(71) Applicant: SHENZHEN SMOORE TECHNOLOGY LIMITED, Shenzhen (CN)

(72) Inventors: Haihua LI, Shenzhen (CN); Zhichao CHEN, Shenzhen (CN); Yue JIANG, Shenzhen (CN); Shaobin XIANG, Shenzhen (CN)

(21) Appl. No.: 18/357,079

(22) Filed: Jul. 21, 2023

Related U.S. Application Data
(63) Continuation of application No. PCT/CN2021/142007, filed on Dec. 28, 2021.

(30) Foreign Application Priority Data
Jan. 27, 2021 (WO) PCT/CN2021/073998

(51) Int. Cl. C04B 35/565; C04B 35/626; C04B 41/00; A24F 40/46

(52) U.S. Cl. CPC C04B 35/565; C04B 35/6261; C04B 35/62655; C04B 41/0072; A24F 40/46; C04B 2235/3826; C04B 2235/3217; C04B 2235/3418; C04B 2235/36; C04B 2235/9607; C04B 2235/3208; C04B 2235/3215; C04B 2235/3409; C04B 2235/3284; C04B 2235/6567

(57) ABSTRACT

A ceramic substrate includes: (a) 10 to 70 wt % of silicon carbide; (b) 6 to 60 wt % of aluminum oxide; (c) 5 to 45 wt % of silicon dioxide; and (d) 0 to 15 wt %, excluding 0, of glass powder. Each component is provided as a raw material. A weight percentage of each component is based on a mass percentage of each component.

### CROSS-REFERENCE TO PRIOR APPLICATION

This application is a continuation of International Patent Application No. PCT/CN2021/142007, filed on Dec. 28, 2021, which claims priority to International Patent Application No. PCT/CN2021/073998, filed on Jan. 27, 2021. The entire disclosure of both applications is hereby incorporated by reference herein.

### FIELD

This application relates to the technical field of ceramic production, and specifically, to a ceramic substrate and a preparation method for the same, a ceramic heating body, and an electronic vaporization device.

### BACKGROUND

An electronic vaporization device has an appearance and taste similar to those of a conventional cigarette, but usually does not include tar, suspended particulates, and other harmful ingredients in the cigarette. Therefore, the electronic vaporization device is commonly used as a substitute for the cigarette.

The electronic vaporization device generally includes a heating body, and currently, a ceramic heating body is widely used. The ceramic heating body includes a ceramic substrate. A sintering temperature is generally increased to improve compressive strength of the ceramic substrate. However, increasing the sintering temperature may reduce a porosity of a material, and the material may become brittle and deteriorate. In addition, the reduced porosity of the material may affect an e-liquid guiding rate, causing insufficient e-liquid supply.

### SUMMARY

In an embodiment, the present invention provides a ceramic substrate, comprising: (a) 10 to 70 wt % of silicon carbide; (b) 6 to 60 wt % of aluminum oxide; (c) 5 to 45 wt % of silicon dioxide; and (d) 0 to 15 wt %, excluding 0, of glass powder, wherein each component is provided as a raw material, and wherein a weight percentage of each component is based on a mass percentage of each component.

### DETAILED DESCRIPTION

In an embodiment, the present invention overcomes a defect in the related art that compressive strength of a ceramic substrate is improved while a porosity and an e-liquid guiding rate are reduced. In view of this, a ceramic substrate and a preparation method for the same, a ceramic heating body, and an electronic vaporization device are provided. The ceramic substrate can improve the compressive strength of the ceramic substrate without reducing the porosity and the e-liquid guiding rate.

In an embodiment, the present invention provides a ceramic substrate, based on a mass percentage of each component, includes the following raw materials:
(a) 10 to 70 wt % of silicon carbide;
(b) 6 to 60 wt % of aluminum oxide;
(c) 5 to 45 wt % of silicon dioxide; and
(d) 0 to 15 wt %, excluding 0, of glass powder.

That not reducing the porosity and the e-liquid guiding rate means that the porosity and the e-liquid guiding rate at least remain unchanged, and optionally, the porosity and the e-liquid guiding rate are increased. An objective of this application is to make the porosity and e-liquid guiding rate not reduced, and improve the compressive strength of the ceramic substrate.

The foregoing content of each component is a mass percentage of each component, that is, a percentage of each component to a total mass of all components, where a sum of mass percentages of components is 100%. Raw materials of the ceramic substrate include silicon carbide, aluminum oxide, silicon dioxide, glass powder, and other components that can be optionally added. The other components may bring additional properties to the ceramic substrate.

The content of the silicon carbide is, for example, 10 wt %, 12 wt %, 15 wt %, 20 wt %, 25 wt %, 30 wt %, 35 wt %, 40 wt %, 45 wt %, 50 wt %, 55 wt %, 60 wt %, 65 wt %, or 70 wt %. When the content of the silicon carbide is excessively high, a sintering temperature may be excessively high, thermal conductivity of the ceramic substrate may be increased, and thermal efficiency of the ceramic heating body is reduced. When the content of the silicon carbide is excessively low, the compressive strength of the ceramic substrate may be reduced. Considering the compressive strength and thermal conductivity of the ceramic substrate, the content of the silicon carbide may optionally range from 20 wt % to 50 wt %.

The content of the aluminum oxide is, for example, 6 wt %, 10 wt %, 15 wt %, 20 wt %, 25 wt %, 30 wt %, 35 wt %, 40 wt %, 45 wt %, 50 wt %, or 60 wt %. When the content of the aluminum oxide is excessively high, a sintering temperature may be increased, increasing the thermal conductivity of the ceramic substrate and reducing the thermal efficiency of the ceramic heating body. When the content of the aluminum oxide is excessively low, the compressive strength of the ceramic substrate may be reduced. Considering the compressive strength and thermal conductivity of the ceramic substrate, the content of the aluminum oxide may optionally range from 10 wt % to 30 wt %.

The content of the silicon dioxide is, for example, 5 wt %, 10 wt %, 15 wt %, 20 wt %, 25 wt %, 30 wt %, 35 wt %, or 40 wt %. When the content of the silicon dioxide is excessively high, the e-liquid guiding rate of the ceramic substrate may be reduced. When the content of the silicon dioxide is excessively low, the compressive strength of the ceramic substrate may be affected. Considering the compressive strength and e-liquid guiding rate of the ceramic substrate, the content of the silicon dioxide may optionally range from 15 wt % to 25 wt %.

The content of the glass powder is, for example, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, or 15 wt %. When the content of the glass powder is excessively high, the e-liquid guiding rate and porosity of the ceramic substrate may be reduced. When the content of the glass powder is excessively low, the compressive strength of the ceramic substrate may be affected. Considering the compressive strength, porosity, and e-liquid guiding rate of the ceramic substrate, the content of the glass powder may optionally range from 5 wt % to 12 wt %.

In an optional implementation of this application, the porosity of the ceramic substrate ranges from 50% to 60%, and the compressive strength of the ceramic substrate ranges from 15 MPa to 45 MPa.

It should be noted that, in this application, for a method for testing the porosity, refer to the GB/T1966-1996 ceramic open porosity and capacity test method, and for a method for testing the compressive strength, refer to the GB/T1964-1996 ceramic compressive strength test method, where a size of a test sample is 9 mm×3.2 mm×2 mm, and a test contact area is 3.2 mm×2 mm.

In an optional implementation of this application, the thermal conductivity of the ceramic substrate ranges from 0.8 w/mk to 2.4 w/mk.

It should be noted that, in this application, a method for testing the thermal conductivity is a Hot Disk transient plane source method.

In an optional implementation of this application, the glass powder includes at least one of silicon dioxide, aluminum oxide, calcium oxide, sodium oxide, potassium oxide, barium oxide, boron oxide, or zinc oxide, and optionally includes silicon dioxide, aluminum oxide, calcium oxide, sodium oxide, potassium oxide, barium oxide, boron oxide, and zinc oxide.

This application further provides a ceramic substrate, based on a mass percentage of each component, including the following components:

10 to 70 wt % of silicon carbide, 6 to 65 wt % of aluminum oxide, 15 to 50 wt % of silicon dioxide, 0.8 to 2.3 wt % of calcium oxide, 0.1 to 0.4 wt % of sodium oxide, 0.1 to 0.2 wt % of potassium oxide, 0.1 to 0.2 wt % of barium oxide, 0.1 to 0.4 wt % of boron oxide, and 0.2 to 0.5 wt % of zinc oxide.

The foregoing content of each component is a mass percentage of each component, that is, a percentage of each component to a total mass of all components, where a sum of mass percentages of components is 100%.

This application further provides a method for preparing the ceramic substrate, including:

sequentially grinding, drying, granulating, and molding mixed components to form a ceramic green compact, and sintering the ceramic green compact.

In an optional implementation of this application, the grinding includes: grinding the mixed components in the presence of water and a grinding medium, and the grinding includes at least one of the following process parameters:

a temperature in a range of 20° C. to 30° C., a grinding time in a range of 5 min to 30 min, and a grinded material to grinding media ratio in a range of 1:1 to 1:2.5.

The temperature for the grinding is, for example, 21° C., 22° C., 23° C., 24° C., 25° C., 26° C., 27° C., 28° C., 29° C., or 30° C. The grinding time is, for example, 6 min, 10 min, 15 min, min, 25 min, or 30 min.

A typical but non-restrictive grinding medium includes any one of an aluminum oxide grinding ball, a zirconia grinding ball, or an agate grinding ball.

For example, the grinding may be performed in a grinder. The grinded material to grinding media ratio refers to a ratio of a mass of a material to a mass of a grinding medium in the grinder, for example 1:1, 1:1.2, 1:1.4, 1:1.6, 1:1.8, 1:2, 1:2.2, 1:2.4, or 1:2.5.

In an optional implementation of this application, a drying temperature ranges from to 90° C. and a drying time ranges from 4h to 8h.

In an optional implementation of this application, a molding pressure ranges from MPa to 40 MPa, for example, 10 MPa, 15 MPa, 20 MPa, 25 MPa, 30 MPa, 35 MPa, or 40 MPa, and a molding time ranges from 5 s to 20 s, for example, 5 s, 8 s, 11 s, 14 s, 17 s, or 20 s.

The molding may be performed through an automatic dry-pressing molding machine.

In an optional implementation of this application, a sintering temperature ranges from 1100° C. to 1700° C., for example, 1150° C., 1200° C., 1250° C., 1300° C., 1350° C., 1400° C., 1450° C., 1500° C., 1550° C., 1600° C., or 1650° C. When the sintering temperature is lower than 1100° C., the ceramic substrate fails to meet a requirement for use due to insufficient sintering and low compressive strength. When the sintering temperature is higher than 1700° C., the ceramic substrate may have a problem of burning bubbles, greatly reducing the e-liquid guiding rate and porosity. A sintering time ranges from 2h to 8h, for example, 2.5h, 3h, 3.5h, 4h, 4.5h, 5.5h, 6h, 6.5h, 7h, or 7.5h. Optionally, the sintering temperature ranges from 1300° C. to 1500° C., and the sintering time ranges from 2h to 4h.

In an optional implementation of this application, the method for preparing the heating body includes:
grinding mixed components by adding water and a grinding medium, where the grinding medium is an aluminum oxide grinding ball, the temperature for the grinding ranges from 20° C. to 30° C., the grinding time ranges from 5 min to 30 min, and the grinded material to grinding media ratio ranges from 1:1 to 1:2.5;
drying the ground powder, where the drying temperature ranges from 60 to 90° C., and the drying time ranges from 4h to 8h;
granulating the dried powder;
molding the granulated powder to form a ceramic green compact, where the molding pressure ranges from 10 MPa to 40 MPa, and the molding time ranges from 5 s to 20 s; and
sintering the ceramic green compact, where the sintering temperature ranges from 1100° C. to 1700° C., and the sintering time ranges from 2h to 8h.

This application further provides a ceramic heating body. The ceramic heating body is configured to heat and vaporize an aerosol-generation substrate when powered on. The ceramic heating body includes a ceramic substrate as described above and a heating body that is arranged on the ceramic substrate and is configured to generate heat when powered on. The ceramic substrate conducts heat generated by the heating body.

In an optional implementation of this application, the ceramic substrate includes a liquid absorbing surface and a vaporization surface opposite to each other. The heating body is arranged on the vaporization surface. The liquid absorbing surface is configured to absorb an aerosol-generation substrate, the vaporization surface is configured to vaporize the aerosol-generation substrate on the ceramic substrate, and the heating body is arranged on the vaporization surface on one side of the ceramic substrate. The aerosol-generation substrate is, for example, e-liquid. The ceramic substrate absorbs the e-liquid, absorbs the e-liquid into the heating body through a capillary force, to vaporize into aerosols.

This application further provides an electronic vaporization device, including:
a ceramic heating body as described above, configured to heat and vaporize an aerosol-generation substrate when powered on, and
a power supply component, where the ceramic heating body is connected to the power supply component, and the power supply component is configured to supply power to the ceramic heating body.

The technical solution of this application includes the following advantages:

In this application, silicon carbide has high thermal conductivity, a low shrinkage rate, and high-temperature stability. However, a sintering temperature of silicon carbide is relatively high. The aluminum oxide may assist in sintering and reduce the sintering temperature. Thermal conductivity may be controlled through coordination between silicon carbide, aluminum oxide, and silicon dioxide of certain content. In addition, silicon carbide, aluminum oxide, and silicon dioxide may form a skeleton of silicon carbide-corundum-mullite ternary phase at a high temperature, bringing high strength to the material. The glass powder is used as an adhesive that liquefies during the sintering and bonds components together after cooling, to improve anti-bending strength of the heating body. The glass powder is melted into a liquid phrase at a high temperature, to promote migration and sintering of aggregate grains. The aggregate grains are bonded together through the liquid phrase, thereby improving a bonding force between the grains and increasing the strength of the material. In addition, since the glass powder is formed into the liquid phrase at a high temperature and is wrapped the aggregate grains around, a pore originally filled by glass powder grains may enlarge with flowing of glass liquid, thereby improving overall e-liquid guide performance of the material.

In addition to silicon dioxide and aluminum oxide, the glass powder in this application optionally includes calcium oxide, sodium oxide, potassium oxide, barium oxide, boron oxide, and zinc oxide. A function of the foregoing components is to control a melting point of the glass powder, promote sintering of grains, and improve bonding strength of a glass phrase and bonding strength between the glass phrase and the aggregate grains, thereby bringing higher strength to the material.

### Embodiment 1

A ceramic substrate includes, based on a mass percentage of each component, the following raw materials: (a) 18% of silicon carbide; (b) 42% of aluminum oxide; (c) 25% of silicon dioxide; and (d) 15% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 10 min, and a grinded material to grinding media ratio is 1:2;
drying the ground powder, where a drying temperature is 80° C., and a drying time is 5h;
granulating the dried powder;
molding the granulated powder to obtain a ceramic green compact, where a molding pressure is 15 MPa, and a molding time is 10 s; and
sintering the ceramic green compact, where a sintering temperature is 1250° C., and the sintering time is 2.5h.

The obtained ceramic substrate, based on a mass percentage of each component, includes the following components:

18 wt % of silicon carbide, 43.2 wt % of aluminum oxide, 34.9 wt % of silicon dioxide, 2.3 wt % of calcium oxide, 0.4 wt % of sodium oxide, 0.1 wt % of potassium oxide, 0.2 wt % of barium oxide, 0.4 wt % of boron oxide, and 0.5 wt % of zinc oxide.

### Embodiment 2

A ceramic substrate includes, based on a mass percentage of each component, the following raw materials: (a) 28% of silicon carbide; (b) 32% of aluminum oxide; (c) 35% of silicon dioxide; and (d) 5% of glass powder.

The method for preparing the ceramic substrate includes:
mixing components, adding water and a grinding medium to grind the mixed components, where the grinding medium is an aluminum oxide grinding ball, a temperature for the grinding is 24° C., a grinding time is 15 min, and a grinded material to grinding media ratio is 1:1.2;
drying the ground powder, where a drying temperature is 70° C., and a drying time is 6h; granulating the dried powder;
molding the granulated powder to obtain a ceramic green compact, where a molding pressure is 16 MPa, and a molding time is 15 s; and
sintering the ceramic green compact, where a sintering temperature is 1450° C., and the sintering time is 3h.

The obtained ceramic substrate, based on a mass percentage of each component, includes the following components:

28 wt % of silicon carbide, 32.3 wt % of aluminum oxide, 38.2 wt % of silicon dioxide, 0.8 wt % of calcium oxide, 0.1 wt % of sodium oxide, 0.2 wt % of potassium oxide, 0.1 wt % of barium oxide, 0.1 wt % of boron oxide, and 0.2 wt % of zinc oxide.

### Embodiment 3

A ceramic substrate includes, based on a mass percentage of each component, the following raw materials: (a) 64% of silicon carbide; (b) 16% of aluminum oxide; (c) 15% of silicon dioxide; and (d) 5% of glass powder.

The method for preparing the ceramic substrate includes:
mixing components, adding water and a grinding medium to grind the mixed components, where the grinding medium is an aluminum oxide grinding ball, a temperature for the grinding is 16° C., a grinding time is 10 min, and a grinded material to grinding media ratio is 1:1.5;
drying the ground powder, where a drying temperature is 75° C., and a drying time is 5h; granulating the dried powder;
molding the granulated powder to obtain a ceramic green compact, where a molding pressure is 15 MPa, and a molding time is 12 s; and
sintering the ceramic green compact, where a sintering temperature is 1500° C., and the sintering time is 3h.

The obtained ceramic substrate, based on a mass percentage of each component, includes the following components:

64 wt % of silicon carbide, 16.3 wt % of aluminum oxide, 18.2 wt % of silicon dioxide, 0.8 wt % of calcium oxide, 0.1 wt % of sodium oxide, 0.2 wt % of potassium oxide, 0.1 wt % of barium oxide, 0.1 wt % of boron oxide, and 0.2 wt % of zinc oxide.

### Embodiment 4

A ceramic substrate includes, based on a mass percentage of each component, the following raw materials: (a) 50% of silicon carbide; (b) 6% of aluminum oxide; (c) 34% of silicon dioxide; and (d) 10% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 10 min, and a grinded material to grinding media ratio is 1:2;
drying the ground powder, where a drying temperature is 80° C., and a drying time is 5h;
granulating the dried powder;
molding the granulated powder to obtain a ceramic green compact, where a molding pressure is 19 MPa, and a molding time is 10 s; and
sintering the ceramic green compact, where a sintering temperature is 1400° C., and the sintering time is 2h.

The obtained ceramic substrate, based on a mass percentage of each component, includes the following components:

50 wt % of silicon carbide, 6.7 wt % of aluminum oxide, 40.7 wt % of silicon dioxide, 1.5 wt % of calcium oxide, 0.3 wt % of sodium oxide, 0.1 wt % of potassium oxide, 0.1 wt % of barium oxide, 0.3 wt % of boron oxide, and 0.3 wt % of zinc oxide.

### Embodiment 5

A ceramic substrate includes, based on a mass percentage of each component, the following raw materials: (a) 50% of silicon carbide; (b) 21% of aluminum oxide; (c) 17% of silicon dioxide; and (d) 12% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 15 min, and a grinded material to grinding media ratio is 1:2;
drying the ground powder, where a drying temperature is 80° C., and a drying time is 4h; granulating the dried powder;
molding the granulated powder to obtain a ceramic green compact, where a molding pressure is 13 MPa, and a molding time is 10 s; and
sintering the ceramic green compact, where a sintering temperature is 1320° C., and the sintering time is 4h.

The obtained ceramic substrate, based on a mass percentage of each component, includes the following components:

50 wt % of silicon carbide, 21.8 wt % of aluminum oxide, 25.0 wt % of silicon dioxide, 1.8 wt % of calcium oxide, 0.4 wt % of sodium oxide, 0.1 wt % of potassium oxide, 0.1 wt % of barium oxide, 0.4 wt % of boron oxide, and 0.4 wt % of zinc oxide.

### Embodiment 6

A ceramic substrate includes, based on a mass percentage of each component, the following raw materials: (a) 20% of silicon carbide; (b) 50% of aluminum oxide; (c) 20% of silicon dioxide; and (d) 10% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 15 min, and a grinded material to grinding media ratio is 1:2;
drying the ground powder, where a drying temperature is 85° C., and a drying time is 6h;
granulating the dried powder;
molding the granulated powder to obtain a ceramic green compact, where a molding pressure is 14 MPa, and a molding time is 15 s; and
sintering the ceramic green compact, where a sintering temperature is 1375° C., and the sintering time is 2.5h.

The obtained ceramic substrate, based on a mass percentage of each component, includes the following components:

20 wt % of silicon carbide, 50.7 wt % of aluminum oxide, 26.7 wt % of silicon dioxide, 1.5 wt % of calcium oxide, 0.3 wt % of sodium oxide, 0.1 wt % of potassium oxide, 0.1 wt % of barium oxide, 0.3 wt % of boron oxide, and 0.3 wt % of zinc oxide.

### Embodiment 7

A ceramic substrate includes, based on a mass percentage of each component, the following raw materials: (a) 55% of silicon carbide; (b) 20% of aluminum oxide; (c) 10% of silicon dioxide; and (d) 15% of glass powder.

The method for preparing the ceramic substrate includes:
mixing components, adding water and a grinding medium to grind the mixed components, where the grinding medium is an aluminum oxide grinding ball, a temperature for the grinding is 26° C., a grinding time is 20 min, and a grinded material to grinding media ratio is 1:2.5;
drying the ground powder, where a drying temperature is 65° C., and a drying time is 8h; granulating the dried powder;
molding the granulated powder to obtain a ceramic green compact, where a molding pressure is 20 MPa, and a molding time is 15 s; and
sintering the ceramic green compact, where a sintering temperature is 1350° C., and the sintering time is 4h.

The obtained ceramic substrate, based on a mass percentage of each component, includes the following components:

55 wt % of silicon carbide, 21.1 wt % of aluminum oxide, 20.0 wt % of silicon dioxide, 2.3 wt % of calcium oxide, 0.4 wt % of sodium oxide, 0.1 wt % of potassium oxide, 0.2 wt % of barium oxide, 0.4 wt % of boron oxide, and 0.5 wt % of zinc oxide.

### Embodiment 8

A ceramic substrate includes, based on a mass percentage of each component, the following raw materials: (a) 55% of silicon carbide; (b) 19% of aluminum oxide; (c) 15% of silicon dioxide; and (d) 11% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 25 min, and a grinded material to grinding media ratio is 1:2;
drying the ground powder, where a drying temperature is 80° C., and a drying time is 5h; granulating the dried powder;
molding the granulated powder to obtain a ceramic green compact, where a molding pressure is 15 MPa, and a molding time is 15 s; and
sintering the ceramic green compact, where a sintering temperature is 1500° C., and the sintering time is 2.5h.

The obtained ceramic substrate, based on a mass percentage of each component, includes the following components:

55 wt % of silicon carbide, 20.0 wt % of aluminum oxide, 22.1 wt % of silicon dioxide, 1.7 wt % of calcium oxide, 0.3 wt % of sodium oxide, 0.1 wt % of potassium oxide, 0.1 wt % of barium oxide, 0.3 wt % of boron oxide, and 0.4 wt % of zinc oxide.

### Embodiment 9

A ceramic substrate includes, based on a mass percentage of each component, the following raw materials: (a) 30% of silicon carbide; (b) 16% of aluminum oxide; (c) 44% of silicon dioxide; and (d) 10% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 10 min, and a grinded material to grinding media ratio is 1:2;
drying the ground powder, where a drying temperature is 85° C., and a drying time is 4h; granulating the dried powder;
molding the granulated powder to obtain a ceramic green compact, where a molding pressure is 25 MPa, and a molding time is 10 s; and
sintering the ceramic green compact, where a sintering temperature is 1450° C., and the sintering time is 5h.

The obtained ceramic substrate, based on a mass percentage of each component, includes the following components:

30 wt % of silicon carbide, 17.2 wt % of aluminum oxide, 49.1 wt % of silicon dioxide, 2.2 wt % of calcium oxide, 0.4 wt % of sodium oxide, 0.1 wt % of potassium oxide, 0.1 wt % of barium oxide, 0.4 wt % of boron oxide, and 0.5 wt % of zinc oxide.

### Embodiment 10

A ceramic substrate includes, based on a mass percentage of each component, the following raw materials: (a) 10% of silicon carbide; (b) 60% of aluminum oxide; (c) 15% of silicon dioxide; and (d) 15% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 25 min, and a grinded material to grinding media ratio is 1:1;
drying the ground powder, where a drying temperature is 90° C., and a drying time is 4h;
granulating the dried powder;
molding the granulated powder to obtain a ceramic green compact, where a molding pressure is 25 MPa, and a molding time is 15 s; and
sintering the ceramic green compact, where a sintering temperature is 1200° C., and the sintering time is 6h.

The obtained ceramic substrate, based on a mass percentage of each component, includes the following components:

10 wt % of silicon carbide, 61.0 wt % of aluminum oxide, 25.1 wt % of silicon dioxide, 2.3 wt % of calcium oxide, 0.4 wt % of sodium oxide, 0.1 wt % of potassium oxide, 0.2 wt % of barium oxide, 0.4 wt % of boron oxide, and 0.5 wt % of zinc oxide.

### Embodiment 11

A ceramic substrate includes, based on a mass percentage of each component, the following raw materials: (a) 70% of silicon carbide; (b) 10% of aluminum oxide; (c) 5% of silicon dioxide; and (d) 15% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 25 min, and a grinded material to grinding media ratio is 1:2;
drying the ground powder, where a drying temperature is 80° C., and a drying time is 7h; granulating the dried powder;
molding the granulated powder to obtain a ceramic green compact, where a molding pressure is 30 MPa, and a molding time is 10 s; and
sintering the ceramic green compact, where a sintering temperature is 1200° C., and the sintering time is 3h.

The obtained ceramic substrate, based on a mass percentage of each component, includes the following components:

70 wt % of silicon carbide, 11.1 wt % of aluminum oxide, 15.0 wt % of silicon dioxide, 2.3 wt % of calcium oxide, 0.4 wt % of sodium oxide, 0.1 wt % of potassium oxide, 0.2 wt % of barium oxide, 0.4 wt % of boron oxide, and 0.5 wt % of zinc oxide.

### Comparative Example 1

A ceramic substrate includes, based on a mass percentage of each component, the following raw materials: (a) 75% of silicon carbide; (b) 1% of aluminum oxide; (c) 4% of silicon dioxide; and (d) 20% of glass powder.

The method for preparing the ceramic substrate includes:
mixing components, adding water and a grinding medium to grind the mixed components, where the grinding medium is an aluminum oxide grinding ball, a temperature for the grinding is 25° C., a grinding time is 10 min, and a grinded material to grinding media ratio is 1:2;
drying the ground powder, where a drying temperature is 80° C., and a drying time is 5h; granulating the dried powder;
molding the granulated powder to obtain a ceramic green compact, where a molding pressure is 20 MPa, and a molding time is 10 s; and
sintering the ceramic green compact, where a sintering temperature is 1250° C., and the sintering time is 3h.

The obtained ceramic substrate, based on a mass percentage of each component, includes the following components: 75 wt % of SiC, 4 wt % of Al₂O₃, 10 wt % of SiO₂, 5 wt % of CaO, 2 wt % of Na₂O, 1 wt % of K₂O, 1 wt % of B₂O₃, 1 wt % of BaO, and 1 wt % of ZnO.

### Comparative Example 2

A ceramic substrate, based on a mass percentage of each component, includes the following raw materials: 55 wt % of silicon carbide, 25 wt % of aluminum oxide, and 20 wt % of silicon dioxide.

The method for preparing the ceramic substrate includes:
mixing components, adding water and a grinding medium to grind the mixed components, where the grinding medium is an aluminum oxide grinding ball, a temperature for the grinding is 25° C., a grinding time is 15 min, and a grinded material to grinding media ratio is 1:1.4;
drying the ground powder, where a drying temperature is 80° C., and a drying time is 4h; granulating the dried powder;
molding the granulated powder to obtain a ceramic green compact, where a molding pressure is 15 MPa, and a molding time is 15 s; and
sintering the ceramic green compact, where a sintering temperature is 1600° C., and the sintering time is 6h. The obtained ceramic substrate, based on a mass percentage of each component, includes the following components: 55 wt % of silicon carbide, 25 wt % of aluminum oxide, and 20 wt % of silicon dioxide.

### Comparative Example 3

Comparative example 3 is a cCELL ceramic vaporization core, and a manufacturer is Dongguan Mike New Material Technology Co., Ltd.

### Comparative Example 4

Comparative example 4 is an FFELM ceramic vaporization core, and a manufacturer is Dongguan Mike New Material Technology Co., Ltd.

### Comparative Example 5

A ceramic substrate, based on a mass percentage of each component, includes the following raw materials: (a) 10% of silicon carbide; (b) 40% of aluminum oxide; (c) 50% of silicon dioxide; (d) 0% of glass powder (in other words, no glass powder is used as a raw material).

The method for preparing the ceramic substrate includes:
mixing components, adding water and a grinding medium to grind the mixed components, where the grinding medium is an aluminum oxide grinding ball, a temperature for the grinding is 25° C., a grinding time is 10 min, and a grinded material to grinding media ratio is 1:2;
drying the ground powder, where a drying temperature is 60° C., and a drying time is 8h; granulating the dried powder;
molding the granulated powder to obtain a ceramic green compact, where a molding pressure is 15 MPa, and a molding time is 15 s; and
sintering the ceramic green compact, where a sintering temperature is 1600° C., and the sintering time is 4h.

The ceramic substrate, according to a mass percentage of each component, includes the following components: 10 wt % of silicon carbide, 40 wt % of aluminum oxide, and 50 wt % of silicon dioxide.

The porosity, e-liquid guiding rate, strength, and thermal conductivity of the ceramic substrate in Embodiments 1-11 and the ceramic substrate in Comparative example 1-5 are tested, and a specific test method includes:

For a method for testing the porosity, refer to the GB/T1966-1996 ceramic open porosity and capacity test method.

For a method for testing the compressive strength, refer to the GB/T1964-1996 ceramic compressive strength test method, where a size of a test sample is 9 mm×3.2 mm×2 mm, and a test contact area is 3.2 mm×2 mm.

In a method for testing the e-liquid guiding rate, a porous ceramic is cut into a neat sample block with a size of 1 cm*1 cm*1 cm, a 10 ml precision injection sampler is used to drop 20-microlitre standard smoke e-liquid (the standard is 50 mg tobacco smoke e-liquid) on a surface of the sample block placed in a horizontal direction, a time required for the liquid to dip into the sample block is observed under an electron microscope, and the e-liquid guiding rate is obtained by calculating a ratio of the standard liquid volume of the e-liquid to the time required for the liquid to dip into the sample block.

A method for testing the thermal conductivity is a Hot Disk transient plane source method.

Performance evaluation of Embodiments 1-11, Comparative examples 1-2, and Comparative example 5 are shown in Table 2:

TABLE 2

| | | E-Liquid | | |
|---|---|---|---|---|
| | Poros- | guiding | | Thermal |
| | ity/ | rate | Strength/ | conductivity |
| | % | μl/s | MPa | w/mk |
| Embodiment 1 | 57 | 1.3 | 28 | 1.3 |
| Embodiment 2 | 57 | 1.36 | 25 | 1.36 |
| Embodiment 3 | 55 | 1.31 | 34 | 2.2 |
| Embodiment 4 | 55 | 1.41 | 43 | 1.5 |
| Embodiment 5 | 55 | 1.6 | 45 | 1.7 |
| Embodiment 6 | 55 | 1.3 | 28 | 1.4 |
| Embodiment 7 | 57 | 1.36 | 24 | 1.8 |
| Embodiment 8 | 55 | 1.8 | 31 | 1.7 |
| Embodiment 9 | 57 | 1.53 | 23 | 1 |
| Embodiment 10 | 55 | 1.2 | 30 | 1.6 |
| Embodiment 11 | 54 | 1.4 | 53 | 2.4 |
| Comparative example 1 | 40 | 0.5 | 12 | 3 |
| Comparative example 2 | 43 | 0.6 | 10 | 2.8 |
| Comparative example 5 | 55 | 1.25 | 20 | 0.8 |

Three cCELL ceramic vaporization cores manufactured by Dongguan Mike New Material Technology Co., Ltd. in Comparative example 3 are tested, the three samples have the same components, and performance evaluation of the three samples are shown in Table 3.

TABLE 3

| | | E-Liquid | | Thermal |
|---|---|---|---|---|
| | Porosity/ | guiding rate | Strength/ | conductivity/ |
| Example | % | μl/s | MPa | w/mk |
| 1 | 55 | 1.15 | 14 | 0.55 |
| 2 | 57 | 1.25 | 13 | 0.5 |
| 3 | 60 | 1.36 | 11 | 0.45 |

Three FFELM ceramic vaporization cores manufactured by Dongguan Mike New Material Technology Co., Ltd. in Comparative example 4 are tested, the three samples have the same components, and performance evaluation of the three samples are shown in Table 4.

TABLE 4

| | | E-Liquid | | Thermal |
|---|---|---|---|---|
| | Porosity/ | guiding rate | Strength/ | conductivity/ |
| Example | % | μl/s | MPa | w/mk |
| 1 | 55 | 1 | 16 | 0.5 |
| 2 | 57 | 1.1 | 14 | 0.45 |
| 3 | 60 | 1.2 | 12 | 0.36 |

By comparing Embodiments 1-11 with Comparative examples 3-4, it can be learned that the porosity in Embodiments 1-11 is the same or close to the porosity in Comparative example 3 and Comparative example 4. However, the strength of the ceramic substrate in Embodiments 1-11 is apparently higher than the strength in Comparative examples 3-4.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A ceramic substrate, comprising:
(a) 10 to 70 wt % of silicon carbide;
(b) 6 to 60 wt % of aluminum oxide;
(c) 5 to 45 wt % of silicon dioxide; and
(d) 0 to 15 wt %, excluding 0, of glass powder,
wherein each component is provided as a raw material, and
wherein a weight percentage of each component is based on a mass percentage of each component.

2. The ceramic substrate of claim 1, wherein a content of the silicon carbide ranges from 20 wt % to 50 wt %.

3. The ceramic substrate of claim 1, wherein a content of the aluminum oxide ranges from 10 wt % to 30 wt %.

4. The ceramic substrate of claim 1, wherein a content of the silicon dioxide ranges from 15 wt % to 25 wt %.

5. The ceramic substrate of claim 1, wherein a content of the glass powder ranges from 5 wt % to 12 wt %.

6. The ceramic substrate of claim 1, wherein a porosity of the ceramic substrate ranges from 50% to 60%, and a compressive strength of the ceramic substrate ranges from 15 MPa to 45 MPa.

7. The ceramic substrate of claim 1, wherein a thermal conductivity of the ceramic substrate ranges from 0.8 w/mk to 2.4 w/mk.

8. The ceramic substrate of claim 1, wherein the glass powder comprises at least one of silicon dioxide, aluminum oxide, calcium oxide, sodium oxide, potassium oxide, barium oxide, boron oxide, and zinc oxide.

9. The ceramic substrate of claim 8, wherein the glass powder comprises silicon dioxide, aluminum oxide, calcium oxide, sodium oxide, potassium oxide, barium oxide, boron oxide, and zinc oxide.

10. A ceramic substrate, comprising:
10 to 70 wt % of silicon carbide;
6 to 65 wt % of aluminum oxide;
15 to 50 wt % of silicon dioxide;
0.8 to 2.3 wt % of calcium oxide;
0.1 to 0.4 wt % of sodium oxide;
0.1 to 0.2 wt % of potassium oxide;
0.1 to 0.2 wt % of barium oxide;
0.1 to 0.4 wt % of boron oxide; and
0.2 to 0.5 wt % of zinc oxide,
wherein a weight percentage of each component is based on a mass percentage of each component.

11. A method for preparing the ceramic substrate of claim 1, comprising:
sequentially grinding, drying, granulating, and molding mixed components to form a ceramic green compact; and
sintering the ceramic green compact.

12. The method of claim 11, wherein the grinding comprises: grinding the mixed components in a presence of water and a grinding medium, and
wherein the grinding comprises at least one of:
a temperature for the grinding in a range of 20° C. to 30° C.,
a grinding time in a range of 5 min to 30 min, and
a grinded material to grinding media ratio in a range of 1:1 to 1:2.5.

13. The method of claim 11, wherein a molding pressure ranges from 10 MPa to 40 MPa, and a molding time ranges from 5 s to 20 s.

14. The method of claim 11, wherein a sintering temperature ranges from 1100° C. to 1700° C., and a sintering time ranges from 2h to 8h.

15. The method of claim 14, wherein a sintering temperature ranges from 1300° C. to 1500° C., and a sintering time ranges from 2h to 4h.

16. The method of claim 11, further comprising:
grinding mixed components by adding water and a grinding medium comprising an aluminum oxide grinding ball, a temperature for the grinding ranging from 20° C. to 30° C., a grinding time ranging from 5 min to 30 min, and a grinded material to grinding medium ratio ranging from 1:1 to 1:2.5, so as to provide a ground powder;
drying the ground powder with a drying temperature ranging from 60 to 90° C. and a drying time ranging from 4h to 8h so as to provide a dried powder;
granulating the dried powder so as to provide a granulated powder;
molding the granulated powder to form a ceramic green compact with a molding pressure ranging from 10 MPa to 40 MPa and the molding time ranging from 5 s to 20 s; and
sintering the ceramic green compact with a sintering temperature ranging from 1100° C. to 1700° C. and a sintering time ranging from 2h to 8h.

17. A ceramic heating body, comprising:
the ceramic substrate of claim 1; and
a heating body arranged on the ceramic substrate and configured to generate heat when powered on,
wherein the ceramic substrate is configured to conduct heat generated by the heating body such that the ceramic heating body is configured to heat and vaporize an aerosol-generation substrate when powered on.

18. The ceramic heating body of claim 17, wherein the ceramic substrate comprises a liquid absorbing surface and a vaporization surface opposite each other, and
wherein the heating body is arranged on the vaporization surface.

19. An electronic vaporization device, comprising:
the ceramic heating body of claim 17; and
a power supply component,
wherein the ceramic heating body is connected to the power supply component, and
wherein the power supply component is configured to supply power to the ceramic heating body.
